(19) United States
(12) Patent Application Publication
(10) Pub. No.: US 2022/0304570 A1
(43) Pub. Date: Sep. 29, 2022

(54) Method and Apparatus for Measuring Vision Function

(71) Applicant: EyeQue Inc., Newark, CA (US)

(72) Inventors: Noam Sapiens, Newark, CA (US); John Serri, Newark, CA (US)

(21) Appl. No.: 16/803,426

(22) Filed: Feb. 27, 2020

Related U.S. Application Data
(63) Continuation-in-part of application No. 16/176,631, filed on Oct. 31, 2018.
Application No. 16/176,631 is a continuation-in-part of application No. 16/276,302, filed on Feb. 14, 2019, now Pat. No. 10,588,507.
Application No. 16/276,302 is a continuation-in-part of application No. 15/491,557, filed on Apr. 19, 2017, now Pat. No. 10,206,566.
(60) Provisional application No. 62/811,492, filed on Feb. 27, 2019.
(60) Provisional application No. 62/579,558, filed on Oct. 31, 2017.
(60) Provisional application No. 62/409,276, filed on Oct. 17, 2016.

(51) Int. Cl. A61B 3/032; A61B 3/02; A61B 3/024; A61B 3/18; A61B 3/06; A61B 3/00

(52) U.S. Cl. CPC A61B 3/032; A61B 3/022; A61B 3/024; A61B 3/18; A61B 3/066; A61B 3/0041

(57) ABSTRACT

A system for replicating a standardized visual acuity test, such as the 20′ Snellen test may comprise a binocular viewer attached to a smartphone. A binocular viewer may comprise a housing comprising a pair tube covers having voids allowing for viewing through a pair of lens tubes with each lens tube in visual communication with a second lens a first lens an aperture and a front cover. The optical systems use an artful combination of front and back lens surfaces, demagnification and other systems to faithfully replicate the sight lines perceived by a user of a traditional 20′ test. The system also allows for the incorporation of other tests conducted with both eyes including Color Sensitivity and Contrast, furthermore by placing a deformable, tunable lens between the second lens and the eye the device serves as an ophthalmic refractometer, allowing a Spherical Equivalent refraction estimate for each eye.

### RELATED PATENT APPLICATION AND INCORPORATION BY REFERENCE

This utility application claims the benefit and priority of U.S. application 62/811,492 filed on Feb. 27, 2019, the contents of which are incorporated herein.

This utility patent application is a Continuation in Part, CIP of U.S. application Ser. No. 16/176,631 Smart Phone Based Virtual Visual Charts for Measuring Visual Acuity filed on Oct. 31, 2018, which claims the benefit of and priority date of provisional patent application 62/579,558 filed on Oct. 31, 2017.

This utility application is a Continuation in Part, CIP of patent application Ser. No. 16/276,302 filed on Feb. 14, 2019 which is a CIP of application Ser. No. 15/491,557 filed on Apr. 19, 2017, now U.S. Pat. No. 10,206,566 issued on Feb. 19, 2019, which claims the benefit of provisional patent application 62/409,276 filed on Oct. 17, 2016.

If any conflict arises between the disclosure of the invention in this utility application and that in the related applications, the disclosure in this utility application shall govern. Moreover, the inventor(s) incorporate herein by reference any and all patents, patent applications, and other documents hard copy or electronic, cited or referred to in this application.

### COPYRIGHT AND TRADEMARK NOTICE

This application includes material which is subject or may be subject to copyright and/or trademark protection. The copyright and trademark owner(s) has no objection to the facsimile reproduction by any of the patent disclosure, as it appears in the Patent and Trademark Office files or records, but otherwise reserves all copyright and trademark rights whatsoever. Trademarks may include “VA101” and “Visual Acuity Tracker” “Visual Acuity Screener”, “Insight” and/or “EyeQue Insight”.

### BACKGROUND OF THE INVENTION

### (1) Field of the Invention

The invention generally relates to visual acuity measurement systems. More particularly, the invention relates to the use of lens systems and nearby to user light sources to optically replicate a standard visual acuity test within the confines of a binocular viewer. Disclosed embodiments include the integration of high resolution smartphones, communication systems, data retrieval systems and other components.

### (2) Description of the Related Art

In the related art, standardized visual acuity tests are well known and typically require a 20-foot distance between the test subject and the eye chart. Such tests work well in dedicated testing spaces, such as an eye doctor's office or a government motor vehicle facility. With the advent of smart phones and other electronic devices, and spending less time outdoors, children are developing myopia at an alarming rate. A shortfall in the prior art is that a parent, teacher or caregiver may want to quickly and economically test a child's visual acuity but have neither the oversized paper eyechart of the prior art nor a clear, properly lit 20-foot space. Moreover, children are not likely to stand still to maintain the required 20-foot distance of a traditional test.

The prior art is replete with shortfalls to the visual health and testing of adults as well. With the high cost of eye exams and the current need to physically travel to an eye care professional, many adults are not getting the eye tests they need. Myopia is an increasing problem and is especially acute in low income populations and worse in low-to-middle income countries.

The prior art does include the use of virtual images for eye tests, one such system is sometimes known as the SPOT Vision Screener by Welch Allyn. The Welch Allyn device is exceptionally expensive and not well suited for use by consumers. The Welch Allyn device fails to leverage the high-resolution screens of present day smart phones. The Welch Allyn device requires a three-foot distance between the device and the test subject, making the device unsuited for self-testing. Thus, there is a serious short fall in the related art and room in the art for the presently disclosed embodiments.

Recently, there has been a plethora of free mobile Apps that claim to measure visual acuity, but in order to duplicate the 20′ Snellen test, the phone screen needs to be far away from the user, making the testing highly inconvenient, and in the case of testing children almost impossible. Also, given the fact that there is no constraint on the distance of the tester from the smart phone in these free Apps the results are highly inaccurate, compared to the forced distant constraints of the presently disclosed embodiments.

### BRIEF SUMMARY OF THE INVENTION

The present invention overcomes shortfalls in the related art by presenting an unobvious and unique combination, configuration of disclosed components that include two sets of lenses with optical properties well suited for optically producing a traditional visual acuity test within the relatively short confines of a binocular viewer. The term “visual acuity” may be defined as the eye's ability to detect fine details at a predefined distance. Disclosed embodiments overcome shortfalls in the art by the artful use and integration of high resolution smart phone screens that a provide finely tuned light source. The integration of high resolution smart phone screens also provides infinite possibilities in the presentation of eye charts or symbols used for eye testing. Moreover, the integration of smartphones facilitates the instant analysis of test results and instant communication and electronic storage of test results.

The present invention provides a self-administered vision test solution, which yields similar results as the prior art vision test performed in a doctor's office. With a disclosed binocular viewer working in conjunction with a smartphone running a specific application, the user can perform a self-administered distance (or near) vision test without additional help. Furthermore the system, comprised of the binocular viewer and the smart phone can also be used to conduct other visual tests including contrast sensitivity, color sensitivity, and refractive error. The present invention also provides a method for a user to manage their eye health by providing referrals to eye care professionals. The invention also provides a means for electronic communication between a user, and/or an eye care professional.

The disclosed embodiments overcome shortfalls in the prior art by the use of demagnification occurring on the back side of the first lens which comprises a concave surface.

The disclosed embodiments overcome shortfalls in the art by providing an economical, compact and self-administered visual acuity test that comports with the limited means of many people. The traditional field test often conducted at 20 feet (or 6 meters) to replicate real life visual needs wherein objects 20 feet away are of real relevance. A person of “normal” vision may be said to have 20/20 vision, meaning that the test subject sees the 20/20 line of optotypes (letters, numbers, tumbling E, etc.) at a 20 foot distance. A test subject with “better than normal” vision will see the 20/15 line of optotypes (smaller size than 20/20 line) at a 20-foot distance, deeming them capable of 20/15 vision. Conversely, a test subject with significantly “less than normal” vision such as 20/200 has vision that is 1/10th that of a person with normal vision or would need the objects 10 times closer to see the same 20/20 line that a person with normal vision sees at 20 ft. Based upon the real world need to see objects at 20 feet with clarity, many visual acuity standards are based on the 20 foot bench mark. Thus, virtually replicating the 20 foot bench mark test is of great utility, so long as such virtual or optical replication tests the viewer's ability to resolve an object subtending at an angular range of 20 feet. The presently disclosed embodiments not only simulate the angular view lines of a 20 foot test, but also improve upon the traditional 20 foot test by use of randomly rotating optotypes, static lighting, immediate test result reporting, test analysis and electronic storage.

Moreover, replicating the standard 20 foot test is of utility in detecting a number of conditions including refraction error, astigmatism, myopia, hyperopia, color blindness, glaucoma, and macular degeneration for example.

By inserting an adjustable lens system between lens (360) and the user, the device also serves as a portable phoropter. The user adjusts the power of the lens to reach best visual acuity. As the light emerging from lens (360) representing the screen is nearly parallel, adjusting the lens system will serve to focus the light on the retina.

Accurate refraction values can be achieved by using an adjustable stokes cylindrical lens pair and adjustable spherical lens to offset astigmatic as well as spherical errors for the tunable lens system. Refractive values are used in determining the refraction correction supplied by devices such as prescription eye glasses.

In particular, myopia is the medical term for the common vision condition known as nearsightedness, in which close vision is sharp, but objects farther away appear blurred. The prevalence of myopia has rapidly increased globally over the last 30 years. There is a substantial risk for vision impairment associated with high myopia, including retinal damage, cataract and glaucoma. Myopia is estimated to affect 27% (1.9 billion) of the world population, in 2010. Myopia is projected to effect 33% (2.6 billion) of the world population by 2020 and 50% (5 billion) of the world's population by 2050, according to a World Health Organization (WHO) myopia report.

The disclosed embodiments are well suited for testing the vision of children as the disclosed binocular viewer may be used in small rooms or crowded conditions where securing an eye chart at exactly 20 feet from a test subject and proper lighting is not practical.

Vision problems currently affect 1 in 4 school-aged children in the US and the ratios are even higher in other countries such as Korea and China. Impaired vision in children can cause life-long learning, emotional and behavioral problems. The American Optometric Association recommends a comprehensive eye exam every one to two years. However, due to the rapid development of a child's eye balls, myopic conditions given this timeframe may not be detected until after they have been progressed to a significant degree. Research studies prove that the progression of myopia in children can be slowed or stopped, resulting in better vision for life. Early detection and intervention is paramount in slowing myopia progression in school-aged children. Thus, the presently disclosed embodiments are necessary in providing a convenient, low cost self-administered and easily accessible methods to monitor vision changes, such as the onset of myopia. The disclosed embodiments have global utility. In under-developed countries, there is a dearth of eye care professionals, making vision screenings unavailable to many. Thus, the disclosed embodiments are crucial in providing, access to self-administered and easy-accessed vision screening tools to test visual acuity as a first step towards treatment.

Currently, distance vision tests are normally performed at a doctor's office, as the first step of the comprehensive eye exam to assess visual acuity. In the prior art, the test subject typically stands at a significant distance, usually 20 ft (or 6 meters), from the visual target. The visual target contains different letters with various sizes (Snellen chart), or different orientations of the letter “E” with varies sizes (tumbling E chart) or different orientations of the letter “C” with various sizes (Landolt C chart). The examiner asks the test subject to identify the letters or the orientations of the letters corresponding to a given line on the chart, with each descending chart line comprising letters of smaller size.

The invention comprises a method for self-administered vision screening, which includes the steps of requesting user information, performing visual acuity tests at distance or near, reporting visual acuity results, and tracking visual acuity changes. The results are instantaneously shown on the smartphone after the test, and are stored on a secured cloud server.

A smartphone is used as a display, to create the visual target. In one embodiment, the visual target is chosen to be the tumbling E chart, where the letter “E” with random orientations including up, down, left and right is displayed. The smartphone is attached to the optical device, in a similar fashion as a smartphone is attached to a virtual reality headset. The optical device comprises a unique lens system, which projects the E chart displayed on the smartphone to a virtual distance of 20 feet (6 meters) for distance vision and 14 inches (35 centimeters) for near vision.

The smartphone generates a visual target with white background and black letters, in a similar appearance as a traditional physical eye chart. However, unlike a printed, static and predictable tumbling E chart of the prior art test, in the present embodiments, the letter E and its orientation is randomly generated by the smartphone during the test. Thus, the sequence of letter E orientations is different for each test, minimizing the memory effect which may skew test results.

In one contemplated method of use, a user looks through the binocular viewer with the smartphone attached and uses finger swiping on the touchscreen of the smartphone to interact with an IOS or Android application. Using the swiping gestures of up, down, left and right, the smartphone application receives user input based on the user's perceived current E orientation displayed on the smartphone. After the test, the smartphone application calculates the visual acuity values and displays the results on the screen. A vision record is created and stored upon a secured cloud server, with a time stamp. Over time, a history of vision tests is created and can be used as a reference for monitoring vision changes.

For users who are already moderately myopic or hyperopic, measuring visual acuity without correction would not be appropriate to measure the efficacy of the user's current correction. Thus, disclosed embodiments allow testers to wear either contact lenses or frame glasses, to verify if their current prescription of correction lenses are appropriate, or in other words, if the correction provided by the contacts or the eyeglasses facilitates improved vision, with 20/20 vision being a benchmark.

In the disclosed database systems, the recorded history of vision test results may be shared with parents or eye care professionals, via emails or alerts, wirelessly, minimizing communication cost and time.

Disclosed embodiments include means and methods of ascertaining a test subject's pupillary distance or PD using the smartphone application.

Disclosed embodiments may measure presbyopia and/or act as a phoropter, with tunable spherical and cylindrical values.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 depicts a front perspective view of a disclosed binocular viewer embodiment

FIG. 2 depicts a rear perspective view of a disclosed embodiment

FIG. 3 depicts a top view of a disclosed embodiment

FIG. 4 depicts a bottom view of a disclosed embodiment

FIG. 5 depicts a left side view of a disclosed embodiment

FIG. 6 depicts a right side view of a disclosed embodiment

FIG. 7 depicts a rear view of a disclosed embodiment

FIG. 8 depicts a front view of a disclosed embodiment

FIG. 9 depicts a rear perspective view of a disclosed embodiment with a smart phone attached

FIG. 10 depicts an exploded view of a disclosed embodiment

FIG. 11 depicts disclosed a face plate and other components

FIG. 12 depicts a disclosed housing and other components

FIG. 13 depicts disclosed components disposed within the binocular viewer

FIG. 14 depicts a tracing of vision ray lines

FIG. 15 depicts a typical distance test

FIG. 16 depicts a disclosed testing system as compared to a traditional system

FIG. 17 depicts a sectional view of a disclosed optical system

FIG. 18 depicts a blurred image of the prior art

FIG. 19 depicts a sharp image by use of a disclosed embodiment

FIG. 20 depicts a graph of lens surface properties

FIG. 21A depicts a front view of a first lens

FIG. 21B depicts a side view of a first lens

FIG. 21C depicts perspective view of a first lens

FIG. 22A depicts a front view of a second lens

FIG. 22B depicts a side view of a second lens

FIG. 22C depicts a perspective view of a second lens

FIG. 23 depicts an eye chart image generated upon a smart phone screen

FIG. 24 depicts a flow chart of information obtained from a disclosed embodiment

FIG. 25 depicts an adjustable lens system for refractive correction and other components

FIG. 26 depicts a disclosed embodiment

FIG. 27 depicts a disclosed lens system

FIG. 28 depicts a representation of binocular vision

FIG. 29 depicts a system of vision measurement and recording

FIG. 30 depicts an eye chart

FIG. 31 depicts an eye chart

FIG. 32A to 32B depict eye chart symbols

FIG. 33 depicts a Pelli-Robson Chart

FIGS. 34A and 34B depict Landot C or tumbling E charts

FIG. 35 depicts a sine-wave grating test

FIG. 36 depicts a comparison between contrast sensitivity and spacial frequency

FIG. 37A to 37C depict Ishihara color vision tests

FIGS. 38A and 38B depict fields of vision

FIG. 39A to 39C depict disclosed lens systems

FIG. 40 depicts a steps of a disclosed method

FIG. 41 depicts a Amsler Grid

FIG. 42 depicts a stereopsis depth perception test

FIG. 43 depicts a stereopsis depth perception test

### REFERENCE NUMERALS IN THE DRAWINGS

100 a disclosed embodiment in general
200 housing
205 window
210 foam padding
220 fastener
222 face insert
225 face tube
227 pin guide
240 PD wheel
242 PD knob
245 tube cover
247 hook
250 lens tube
253 pinion gear
254 PD gearing
255 aperture
257 gear cover
260 front cover
265 micro suction tape
300 lens system in general
310 proximal or near eye point of sight rays
320 a first lens
325 first surface or front surface comprising a aspherical surface of a first lens 320
330 second surface or back surface comprising a concave surface of a first lens 320
360 a second lens or spherical convex lens
380 distal or far eye point of sight rays
400 smart phone or other personal electronic device
405 display or screen surface of smartphone
410 strap to secure smart phone to housing
500 eye chart
600 human eye
620 eye lens
640 retina
700 cloud storage/communication system
720 database of user information
740 database for eye care professional
760 database for production of eyeglasses
800 adjustable lens system for refractive correction and other functions

These and other aspects of the present invention will become apparent upon reading the following detailed description in conjunction with the associated drawings.

### DETAILED DESCRIPTION OF EMBODIMENTS OF THE INVENTION

The following detailed description is directed to certain specific embodiments of the invention. However, the invention can be embodied in a multitude of different ways as defined and covered by the claims and their equivalents. In this description, reference is made to the drawings wherein like parts are designated with like numerals throughout.

Unless otherwise noted in this specification or in the claims, all of the terms used in the specification and the claims will have the meanings normally ascribed to these terms by workers in the art.

Unless the context clearly requires otherwise, throughout the description and the claims, the words “comprise,” “comprising” and the like are to be construed in an inclusive sense as opposed to an exclusive or exhaustive sense; that is to say, in a sense of “including, but not limited to.” Words using the singular or plural number also include the plural or singular number, respectively. Additionally, the words “herein,” “above,” “below,” and words of similar import, when used in this application, shall refer to this application as a whole and not to any particular portions of this application.

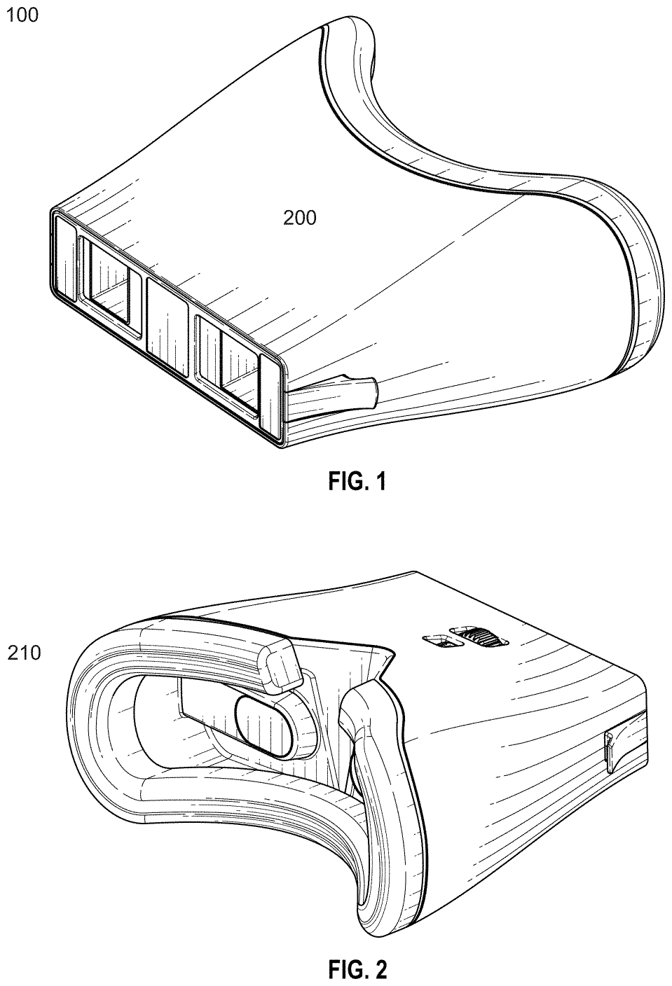

FIG. 1 depicts a disclosed embodiment 100, sometimes referred to as the EyeQue Insight™, optical device or binocular viewer. In general, the disclosed embodiments provide compact, portable and economic means to replicate a standard vision test. In a standard vision test, a test a subject is positioned 20 feet from a physical eye chart. Using the disclosed embodiments, the same experience and test results are replicated by use of a binocular viewer and smartphone. Unlike the prior art, the presently disclosed embodiments seamlessly integrate with electronic storage media, such as cloud systems. In general, disclosed components are encased in a housing 200.

FIG. 2 depicts a perspective view showing a strip of foam padding 210 in the foreground.

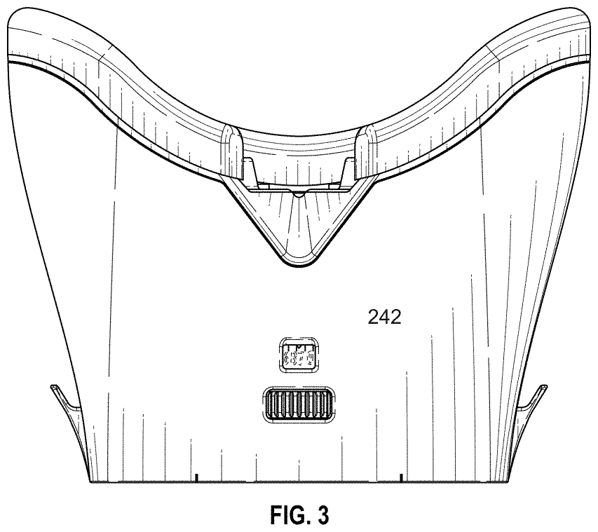

FIG. 3 depicts a top view showing a PD knob 242 used to set the user's estimated pupillary distance or PD. By viewing indicia displayed upon a smart phone, the disclosed embodiments allow a user to rotate the PD knob 242 to align the interval between the barrels to the user's PD. The measured PD is displayed upon the PD wheel 240.

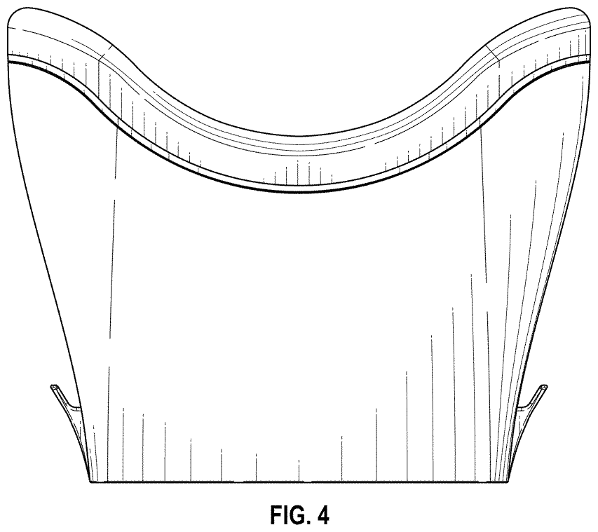

FIG. 4 depicts a bottom side view of a disclosed embodiment.

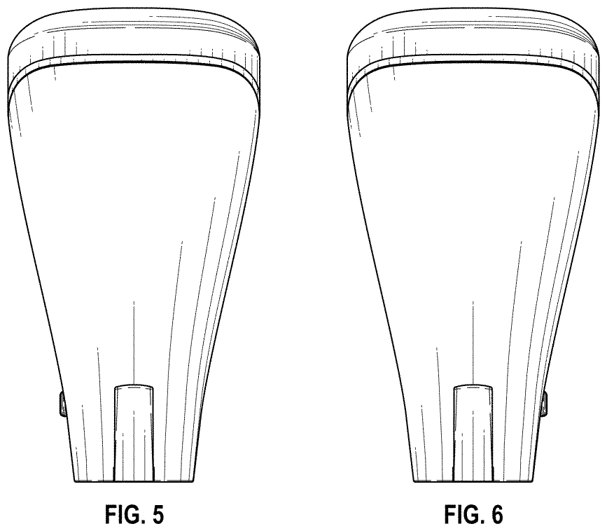

FIG. 5 depicts a right side view and FIG. 6 depicts a left side view.

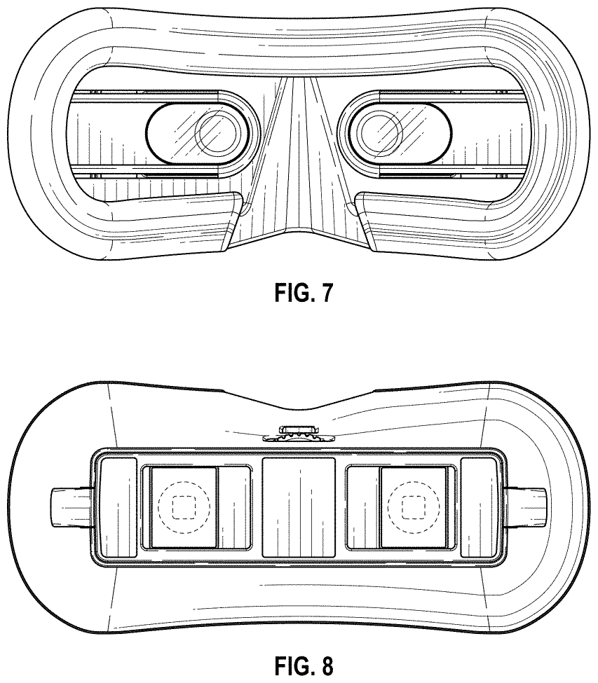

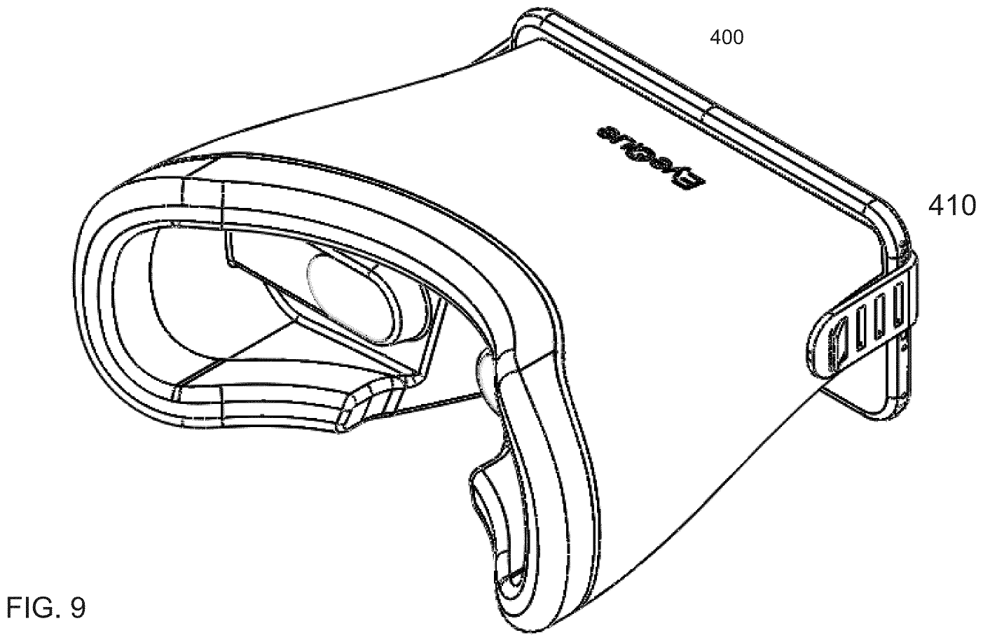

FIG. 7 depicts a front view and FIG. 8 depicts a back view.

FIG. 9 depicts a front and side perspective view with a smart phone 400 or other personal electronic device attached to the back side of the device. A strap 410 or other fastener may be used to secure the phone to the housing.

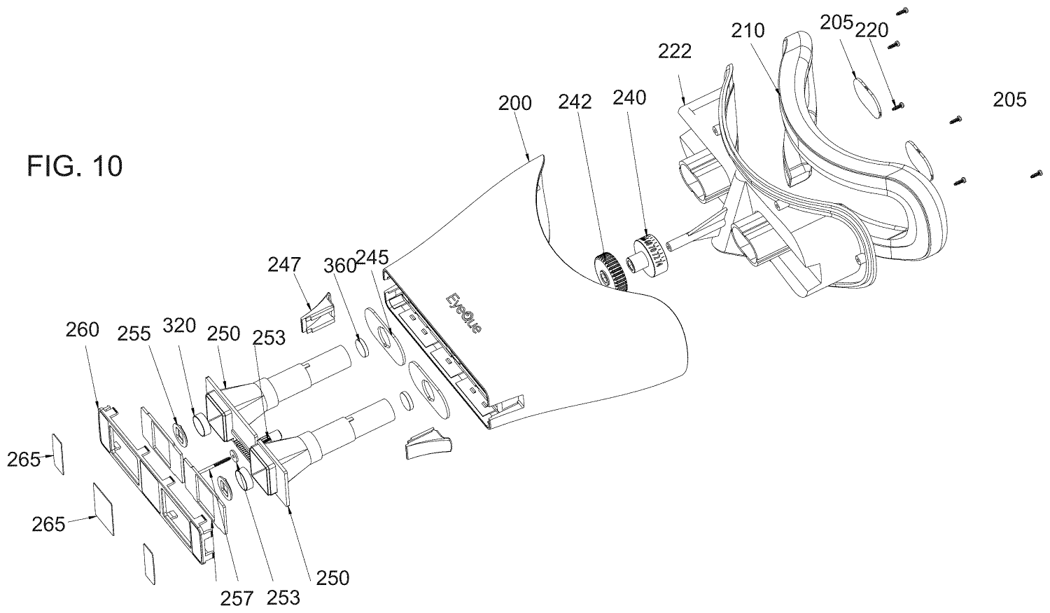

FIG. 10 depicts an exploded view of a disclosed embodiment which may comprise a lens system 300 comprising a first lens 320 or first set of lenses and a second lens 360 or second set of lenses. In general, the lens system optically simulates the prior art vision test requiring 20 feet of space by use of a system requiring less than 11 or so inches. The first and second sets of lenses are secured within lens tubes 250, with the lens tubes moved along the horizontal plane to comport with the user's PD or estimated PD. The user's PD is acquired by the presentation of images upon a smart phone with the distance between the tubes adjusted to the user's PD. A PD knob 242 may be adjusted by the user and the derived PD value or estimated PD value may be observed by viewing the PD wheel.

Starting from the eye of a user or in a proximal position, a window 205 may comprise a transparent flat surface which keeps debris out of the system. Fasteners 220 may attach a face insert 222 upon the housing 200. The windows 205 may be disposed upon or within the face insert and the windows may be centered or aligned to face tubes 225 with the face tubes aligning to a respective lens tube 250.

A pin guide 227 may be disposed upon the face insert 222, with the pin guide axially connected through the PD wheel 240 and PD knob 242. The exterior ends of the face tubes may be aligned within the voids defined by the tube covers 245. The voids defined by the tube covers 245 may be aligned to or may help retain the first set of lenses. The second set of lenses 360 may be retained in or aligned to the proximal ends of the lens tubes 250. The distal ends of the lens tubes may retain or be aligned with the first set of lenses 320. Aperture pieces 255 may define aperture voids with the aperture voids aligned to the first set of lenses 320. A gear cover 257 may be secured to the distal ends of the tubes and a front cover 260 may be secured over the gear cover and within or upon the housing 200. Micro suction tape 265 or other types of fasteners may be applied to the distal side of the front cover 260, with the distal side of the front cover having a planar finished surface to comport to the planar surface of a screen of a smart phone or other electronic device.

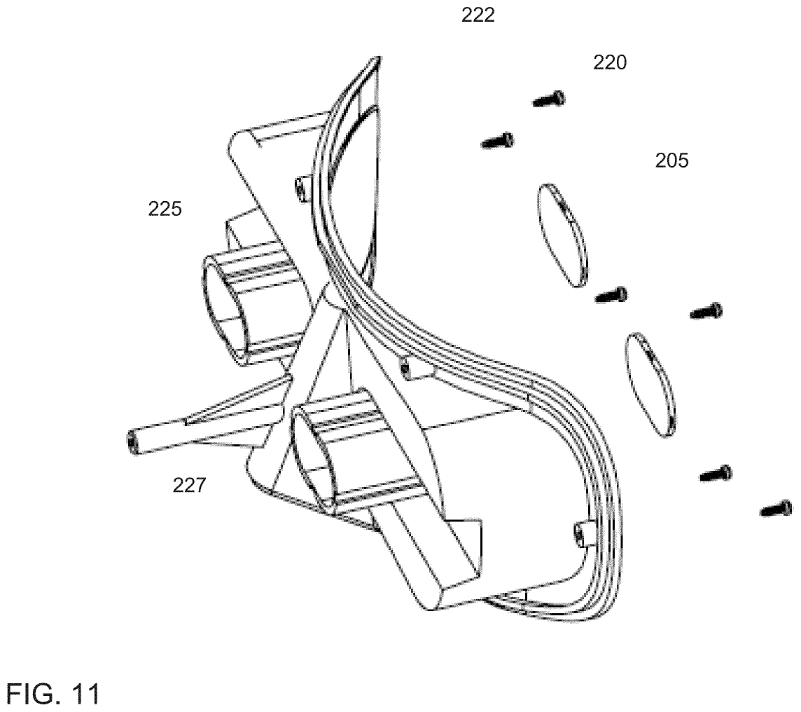

FIG. 11 depicts an expanded view of a face insert 222 and related components.

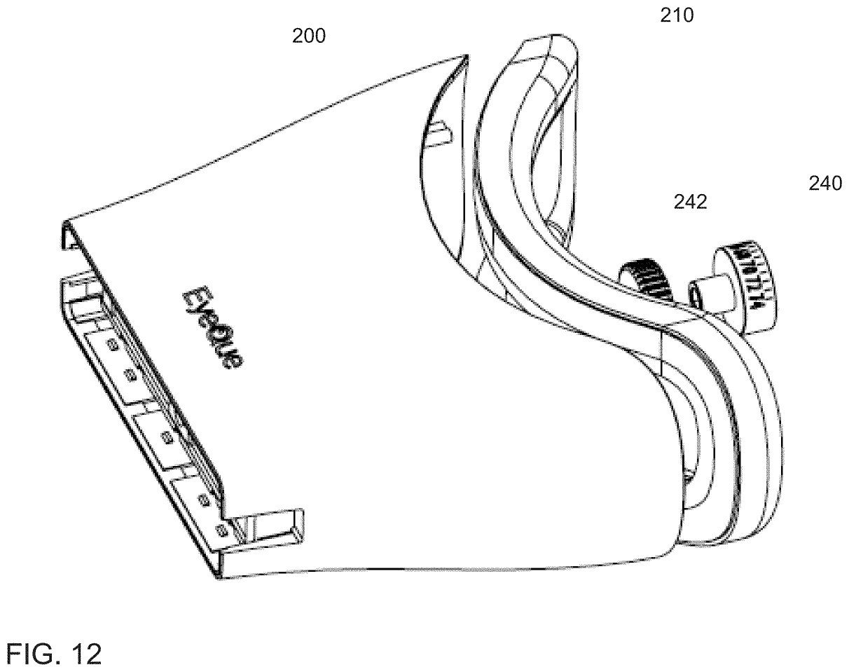

FIG. 12 depicts an expanded view of a housing, strip of foam padding 210, PD knob 242 and PD wheel 240. The PD wheel may comprise markings or indicia indicating a PD obtained or estimated PD in reaction to user adjustments of the PD knob 242.

FIG. 13 depicts an expanded view of the more distal components of the disclosed embodiments.

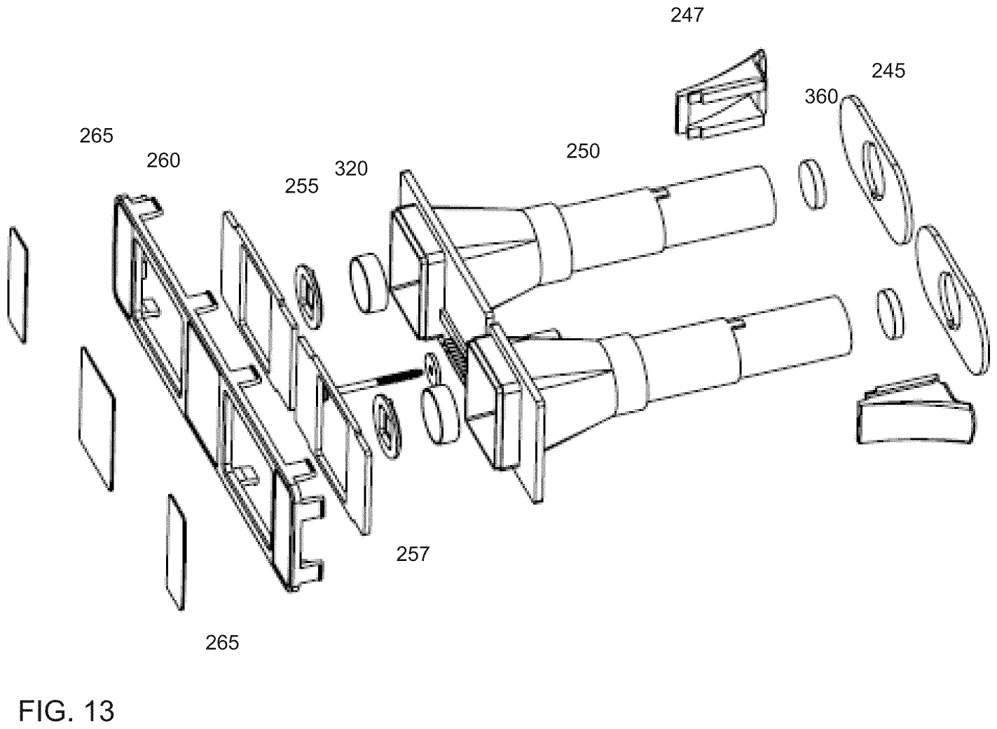

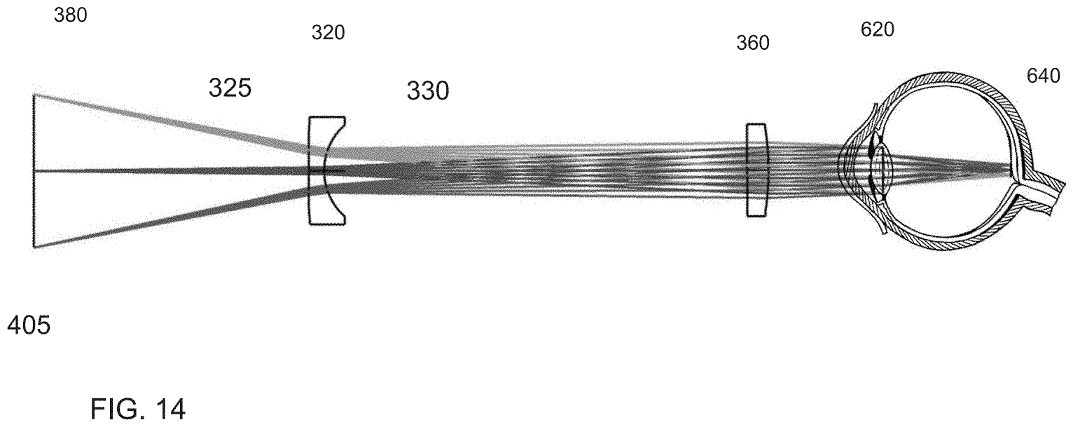

FIG. 14 depicts sight lines or sight rays obtained by a disclosed lens system. Sight rays may start upon or be generated by screen surface 405 of a smart phone. The sight lines or a smart phone image may enter an aspherical surface 325 of a first lens 320. Light will then enter a concave surface 330 of the first lens. Demagnification occurs as a result of the first lens, enabling the production of optically presented optotypes, with the optotypes having the same sight lines as optotypes presented in physical paper form at 20 feet.

The image or light then enters a second lens 360, the second lens comprising a spherical convex lens. The image or light then enters eye lens 620 and then the retina 640.

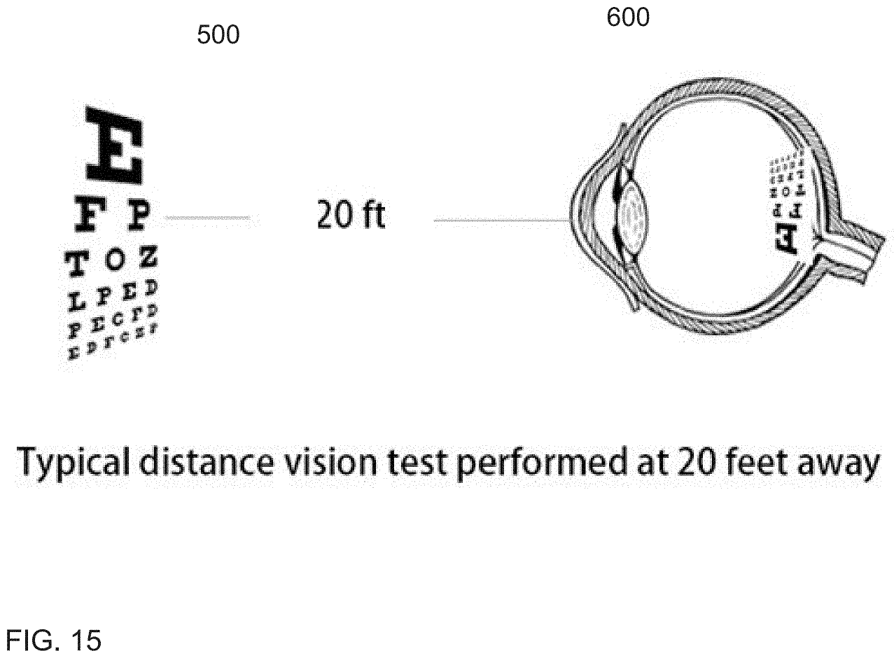

FIG. 15 depicts a typical distance vision test wherein the subject and eye chart are at a distance of 20 feet.

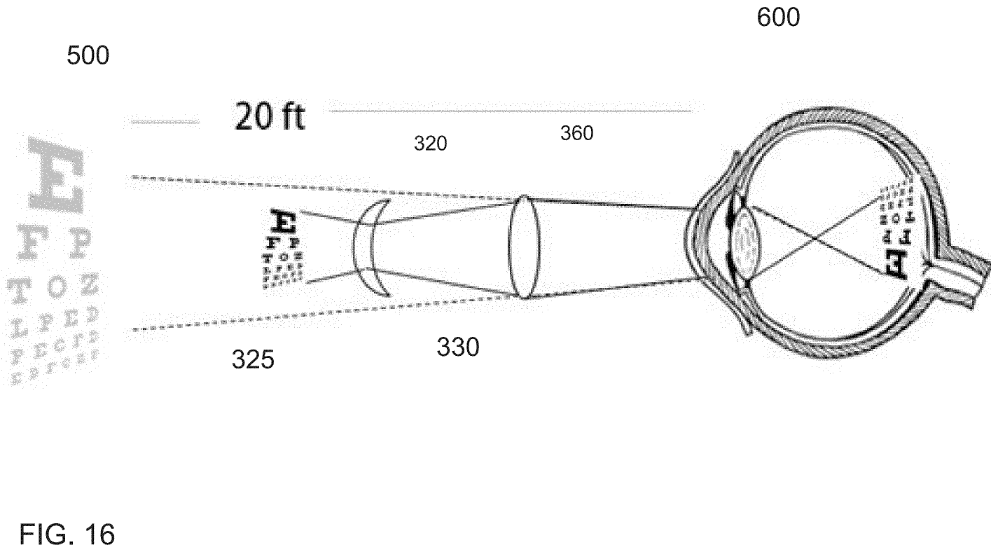

FIG. 16 depicts a comparison of the traditional eye test at 20 feet to the optics of a disclosed embodiment. The artful combination of the first 320 with the second lens 360 creates compact and portable visual acuity test system achieving the same results as the 20 foot test of the prior art. Thus, the images viewed from a disclosed embodiment have the same optical qualities of images viewed in the prior art 20 foot vision test.

FIG. 17 depicts a first lens 320 or lens near the smartphone screen, with the first lens having a first or front side 325 comprising an aspherical surface. The first lens 320 may have a back side comprising a concave surface. A second lens 360 may comprise a spherical convex lens.

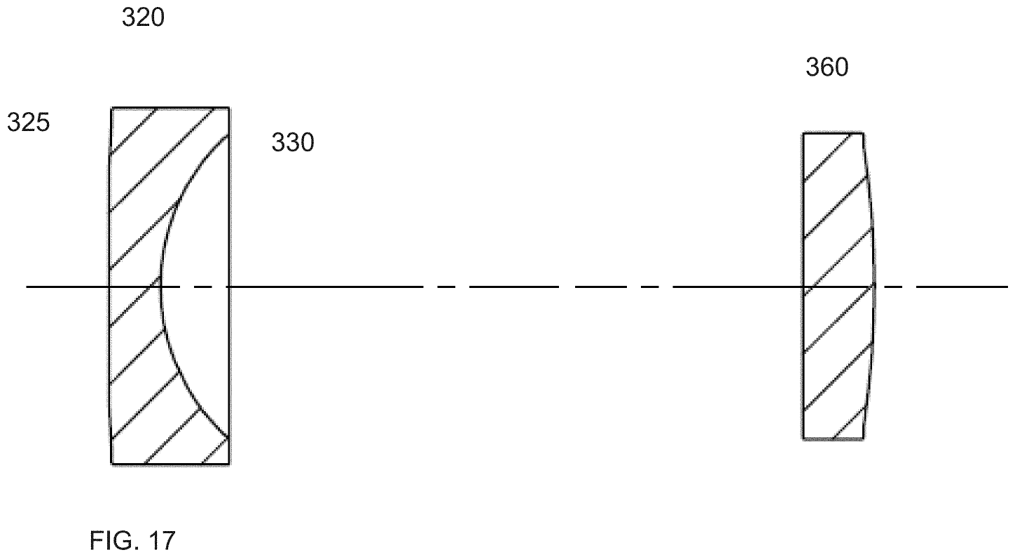

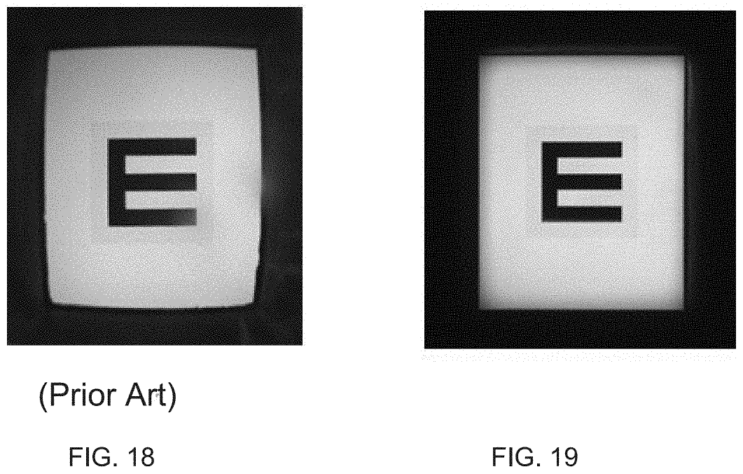

FIG. 18 depicts a barrel distortion of the prior art. The disclosed use of the front aspherical surface of the first lens helps to reduce the barrel distortion of the prior art.

FIG. 19 depicts a more clear view derived by use of a disclosed embodiment.

In a disclosed embodiment, a first lens 320 has a front surface 325 comprising of an aspherical surface, with the aspherical surface used to reduce the optical distortion, such as the barrel effect, observed by a subject using a disclosed embodiment 100. Optical distortion may be considered an optical aberration that deforms and/or bends sight lines, resulting in a curvy or blurred image as exemplified in FIG. 18. The image of FIG. 18 was obtained by use of lenses with spherical surfaces wherein barrel distortion is especially visible along the four outer edges of the image. The four outer straight edges appear to curve as if compressed within a barrel. This phenomenon is sometimes referred to as “barrel distortion.” The disclosed embodiments overcome the barrel distortion of the prior art by use of the disclosed lens system 300 wherein superior results are obtained, as exemplified in FIG. 19.

By use of the disclosed embodiments, shortfalls in the prior art are overcome, such as the short fall of barrel distortion and the short fall of requiring a 20 foot distance between the test subject and the eye chart. The superior results of the disclosed embodiments, as shown in FIG. 19 include significantly reduced barrel distortion wherein the four outside edges appear to be straight or nearly straight.

In the prior art, conventional lenses are made with spherical surfaces. Spherical lenses are known to introduce optical aberrations, such as barrel distortion. A single surface of aspherical profile can greatly reduce the aberration, compared to using a complex spherical lens group. In some of the presently disclosed embodiments, the first surface 325 of the first lens 320 is made with an aspherical profile, meaning that the radius of curvature is not constant across the diameter. A material function of the aspherical surface is to reduce optical distortion and to reproduce the same clear image as viewed from a prior art eye chart at a distance of 20 feet. The second surface 330 of the first lens 320 has a concave spherical profile. The first lens 320 provides a demagnified optical power to generate a virtual image that is approximately three times smaller than the image displayed upon the screen of a smartphone.

The second lens 360 may comprise a spherical convex lens. The second lens 360 creates yet another virtual image or optical image from the first virtual image or optical image created by the first lens 320, at a distance of 20 feet away from the eye. The second lens 360 may have a magnifying optical power of approximately 100.

Overall, a disclosed optical system may have a magnification of around 30. Thus, the letter size displayed upon and by the attached smartphone is about 30 times smaller compared to the letter size of a prior art paper eye chart used for a 20 foot vision test.

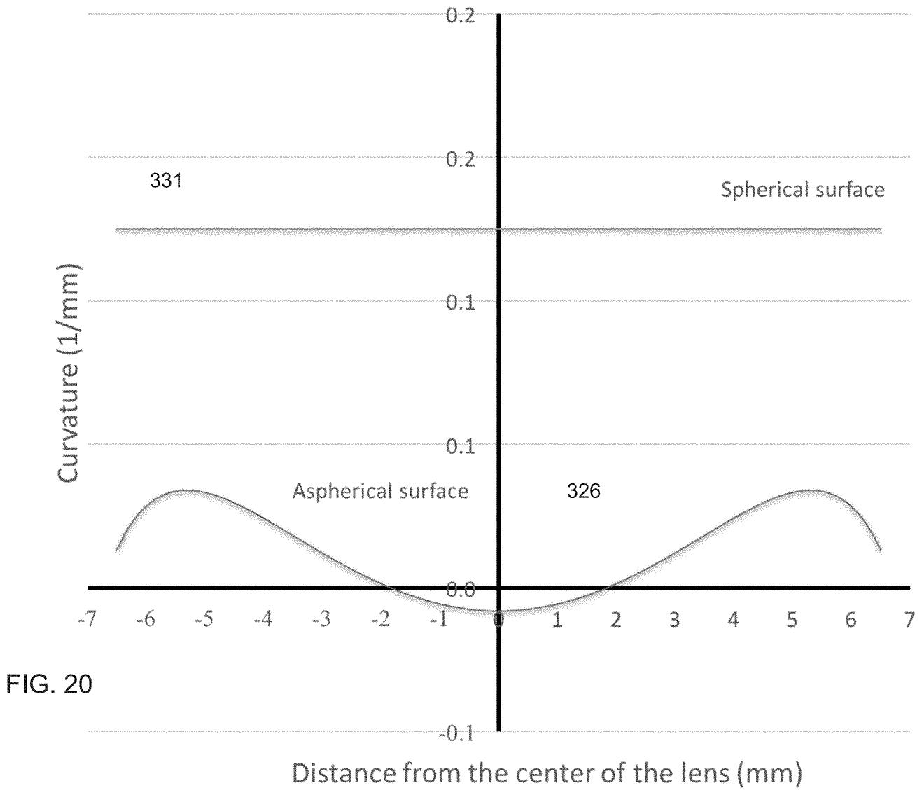

FIG. 20 discloses the best mode known to date for implementing the aspherical surface 325 of the first lens 320. The curved line 326 depicts the curvature value of the aspherical surface, the first surface 325. The straight horizontal line 331 depicts the curvature value of the spherical surface, or second surface 330 of the first lens. The horizontal x axis measures distance in millimeters from the center of a lens while the vertical y axis measures lens curvature in millimeters.

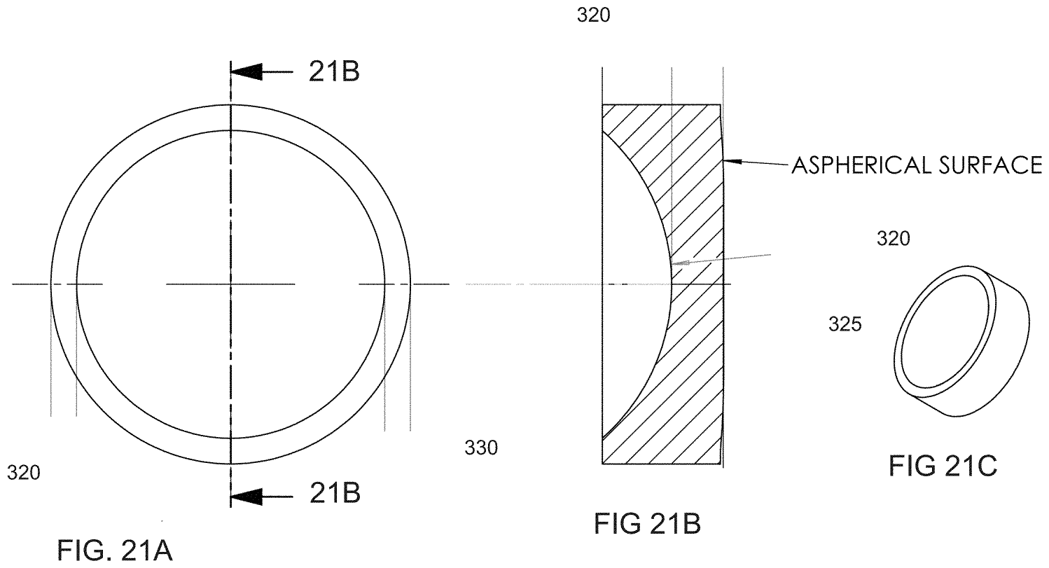

FIG. 21A depicts a front view of a first lens. The first lens may have an outer diameter of 14 mm and an inner diameter of 12 mm.

FIG. 21B depicts a cross sectional view of FIG. 21A. FIG. 21B shows the aspherical surface 325 of the first lens and also shows the concave back surface 330 of the first lens. The outer distance may be 4.71 mm with an inner distance of 2 mm.

FIG. 21C depicts a perspective view of the first lens.

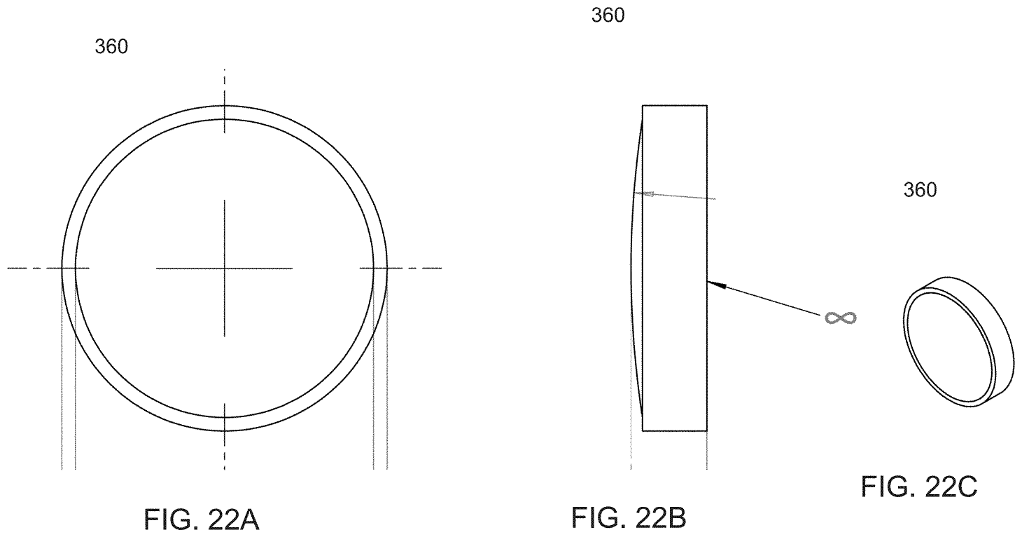

FIG. 22A depicts a front view of a second lens 360 which may have an outside diameter of 12 mm and inside diameter of 11 mm.

FIG. 22B depicts a side view of a second lens wherein the second lens may have a width of 2.8 mm.

FIG. 22C depicts a perspective view of a second lens 360.

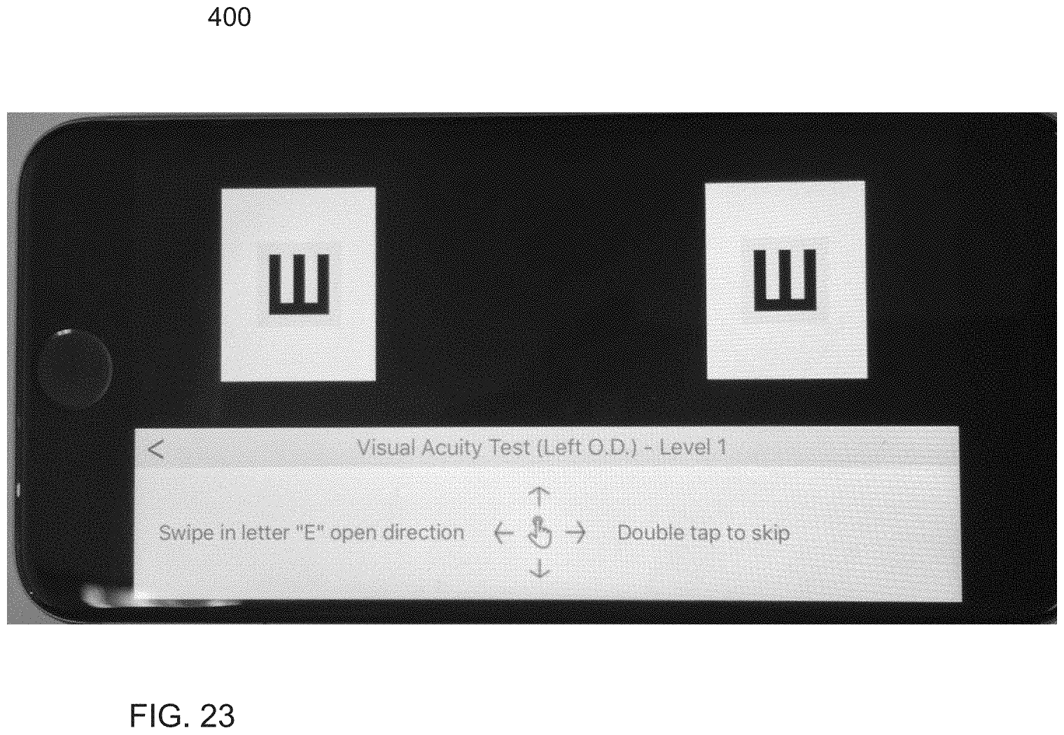

FIG. 23 depicts an image such as an “E” displayed upon a smart phone screen.

FIG. 24 depicts a flow chart of information flowing from a disclosed embodiment 100 to a cloud storage 700 or communication system with the collected data stored or used by a plurality of database systems or outside systems that may include a user measurement database 720, an eye care professional database 740 and a eyeglass production facility database 740.

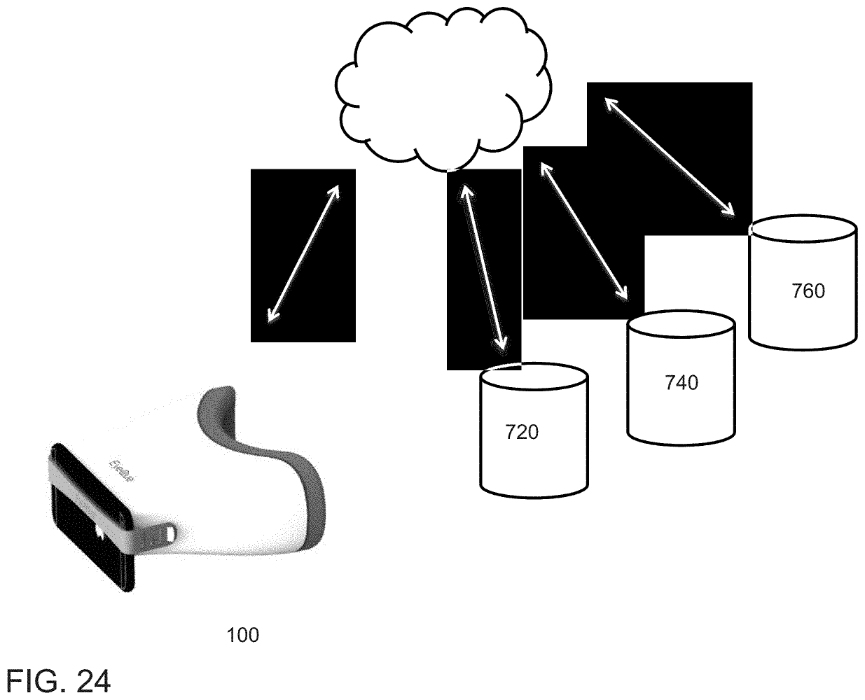

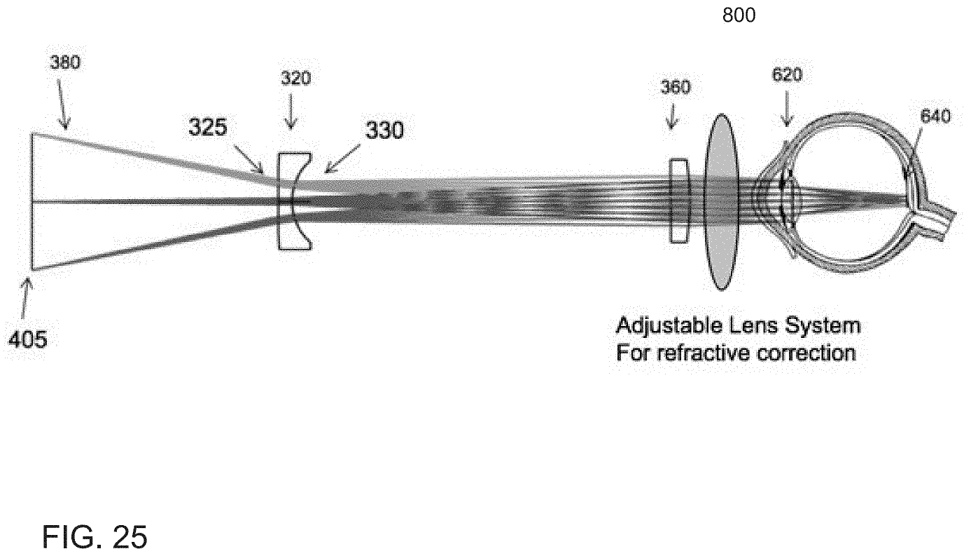

FIG. 25 depicts lenses and sightlines with the addition of an adjustable lens system 800 for refractive corrections and other functions.

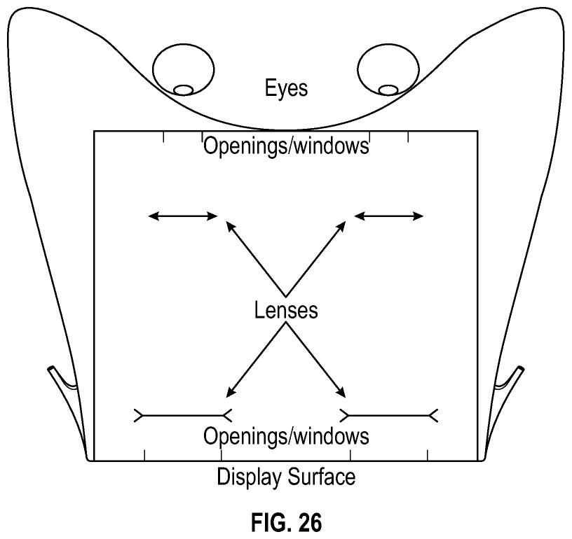

Referring to FIG. 26, a disclosed embodiment based on a binocular viewer device that allows the projection of the test images into the subject's eyes. It enables the projection of an individual and potentially different images to each one of the subject's eyes. The display used to generate the images could be a smartphone display to which the device is attached or alternatively, a screen built into the device. For example, a liquid crystal display (LCD) could be built into the device in the object plane of the optical system. Alternatively, an OLED, spatial light modulator (SLM) or LED array may be used for projecting the images.

In an embodiment of the invention, the device is made of two optical trains as presented in FIG. 26, one for each eye.

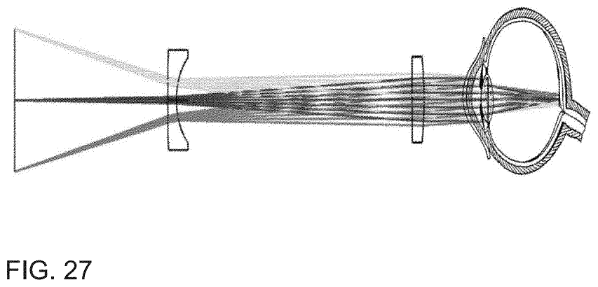

FIG. 27 presents a disclosed optical train. In this implementation of the invention the images from the display are projected onto the subject retina by means of a dual lens set. The lenses are built such that along with the optical system of the subject's eye, the retinal image plane is conjugate with the display used to generate the test images. In one example embodiment of the invention, light from the display is further diverged through the first lens and then converges through the second lens. The arrangement generates a parallel beam at different angular direction corresponding to the different field points on the display. As these beams are incident on the cornea and going through the pupil, they converge on the retina to form a de-magnified image of the display on the retina.

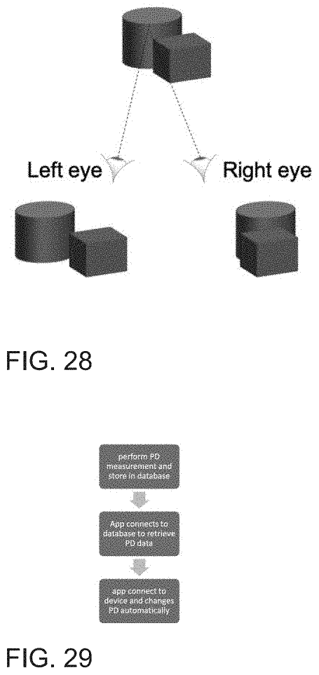

Referring to FIG. 28, The binocular construction of the device allows for depth perception and 3D vision. This could be implemented by “tricking” the human vision system, the visual cortex in the brain, to perceive depth by utilizing the relation between that perception and stereoscopic vision and vergence. Stereopsis (depth perception by stereoscopic vision) is based on disparity in the horizontal direction between the images of the two eyes. As a person focuses on an object, the eyes converge to place that object in the center of the field of view. Therefore, the images on the left eye and the right eye differ due to angular disparity for surrounding objects. As the receptive fields differ due to this horizontal angular disparity, binocular cells in the visual cortex detect the differential and the brain associates it with depth. FIG. 28 shows the images each eye sees with a focus object and another object in front of it.

The expected minimum horizontal disparity that could be detected by 97.5% of the population is 2.3 arcmin, whereas 80% of the population could even detect disparities down to 30 arcsec.

Stereopsis can be segmented into two aspects: coarse and fine. Coarse stereopsis is usually associated with peripheral vision and is responsible for general immersion of a person in the environment. It mainly focuses on dynamic and low spatial frequency objects. Fine stereopsis allows one to determine the depth of an object in the central vision area. It enables the visual cortex image fusion between the images of the two eyes to allow for a coherent 3-dimensional image to be perceived.

Referring to FIG. 29, For the purpose of preventing double vision, allowing for reconciliation of the separate images for each eye in the brain as a single image and for improved vision quality in the device; the device allows for mechanical adjustment of the user's pupillary distance. The mechanism may be manual (e.g. turn wheel and gears, sliders) or automatic (e.g. using a motor). The images on the display need also be adjusted for that distance allowing the center of the FoV to be directly in line with the center of the user's pupils, as is their optical axis. The input to the pupil distance could be an external measurement with a manual input or an automatic one through an application (FIG. 29).

The tests require input from the user in various forms. This could be achieved by using the touch screen of a smartphone or by using controls on the device itself or by using an external controller.

The device could also incorporate a variable lens system to allow for refraction correction. In one embodiment of the invention, the lens could replace the lens that is closest to the use's eye. In another implementation the variable lens could be added to the device between the user's eye and the first lens of the device. In another implementation of the device the lens could be implemented in another location as space permits in the device. The optical design and correction in that case would require additional calibration or calculation to allow for the difference between the actual user eye glass numbers or prescription and the power of the variable lens. The power would depend on the lens location.

The variable lens could be constructed in multiple ways. In an embodiment of the invention the lens could be a liquid lens. In other embodiments of the device the lens could be based on the variable lenses presented herein and related patent applications that have been incorporated herein by reference. Yet another implementation of the variable lens in an embodiment of the invention may be a combination of a zoom lens with a Stokes pair for controlling the cylinder and axis (astigmatism).

### Description of Vision Tests and Example Implementations

Visual Acuity

There are multiple VA test that could be used for assessing a person's vision.

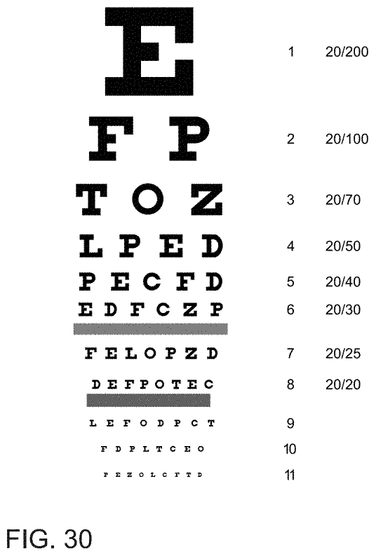

The most prevalent is the Snellen test (FIG. 30).

Further referring to FIG. 30, Letters in each row correspond to a 5 arcmin in the prospective distance on a standard health retina. The line thickness of each letter is designed to be 1 arcmin. The expected healthy human eye resolution is between 30 arcsec and 1 arcmin. The Snellen chart usually uses the 20 fractional notation (also called the Snellen notation), with 20/20 is normal vision, i.e. what a person with normal vision will see at 20 ft. Similarly for example 20/50 is an equivalent of what a normal vision person would see at 50 ft, seen at 20 ft. in this case the size of the letters in that line would correspond to the size of 5 arcmin at 50 ft. Alternative, notations include the metric version which is a 6 base fractional indicating 6 meters instead of 20 ft; log MAR notation which is the logarithm in base 10 of the minimal angular resolution (MAR), which corresponds to the actual angular substance of the symbols on the chart.

The Snellen chart has significant disadvantages resulting from its inherent design.

There are different number of letters per line making the scoring non-standardized.

Letters have various legibility (e.g. D,C,O are easier to read than A,J,L).

Distance between the letters is not standardized and could lead to crowding (the contour interaction between letters that makes it harder to read).

Lack of font standardization—different manufacturers could use different fonts for the charts.

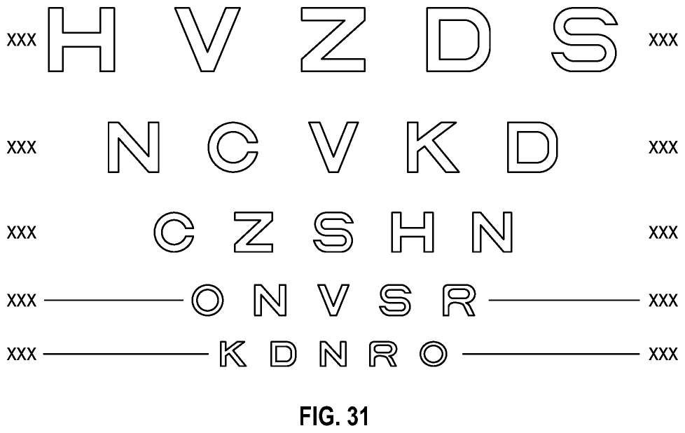

Referring to FIG. 31, A few alternatives were developed including the ETDRS Early Treatment Diabetic Retinopathy Study, currently used as the golden standard by FDA and shown in many studies to have a much higher accuracy level. Yet, caution should be taken when comparing the ETDRS and the Snellen results as is was shown that the ETDRS improves VA by 0.2 log MAR and even more for lower level vision).

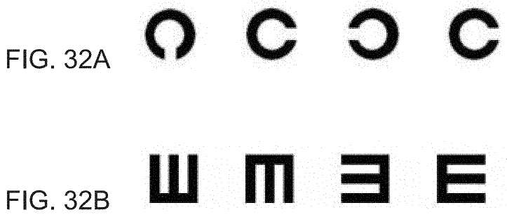

Referring to FIGS. 32A and 32B, Landolt C (FIG. 32A) test and the illiterate/tumbling E (FIG. 32B) tests were developed as a more standardized form of VA tests.

Any of these tests may be used in the device proposed earlier for VA testing. In an embodiment of the invention the user is presented with decreasing size of a tumbling E and requested to indicate which direction the open end of the letter faces. The indication could be done by swiping in that direction on the smartphone screen or by using a separate controller with appropriate buttons for example. Another form of indication could include speech recognition, where the application gets the input by deciphering the user's spoken answer. In this type of input, assuming it is reliable enough, more conventional VA tests could be utilized where the patient reads the letters shown on the display.

Contrast Sensitivity

Contrast sensitivity is a person's ability to distinguish between lighter and darker shades. Contrast sensitivity is a very important measure of visual function. It indicates one's capability to separate objects in various conditions e.g. low light, fog, glare. Driving at night is a prominent example where contrast sensitivity is an important measure. Even if one has 20/20 visual acuity, they can have eye or health conditions that may diminish their contrast sensitivity and make them feel that you are not seeing well. Low contrast sensitivity is indicative of various eye conditions for example cataract and retinal pathologies associated with macular pigment optical density (MPOD).

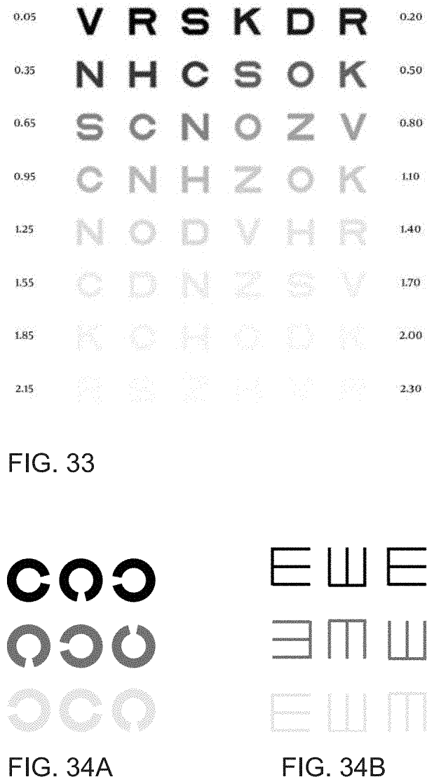

A contrast sensitivity test measures your ability to distinguish between finer and finer increments of light versus dark (contrast). The most common contrast sensitivity test utilized is the Pelli-Robson Chart (FIG. 33). Similar to a VA test, the subject is requested to read letters from the chart, where different lines correspond to lower and lower contrast.

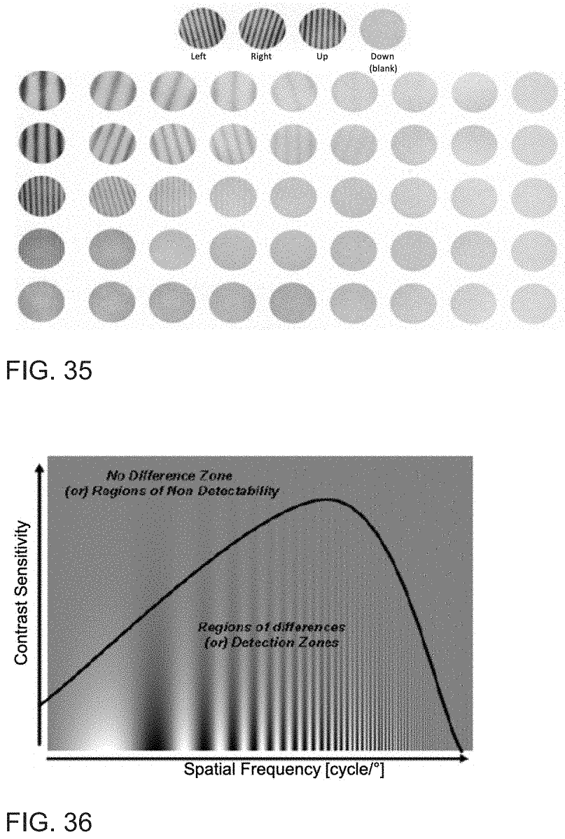

This test could be implemented also using Landot C or tumbling E (FIGS. 34A and 35B)

An example implementation would be such that the letters are presented to the user one at a time (also single eye at a time and for both eyes together). The user would then be requested to indicate the direction of the open end of the letter. The letters would then be shown with reduced contrast. The implementation in this invention has the advantage of optimal lighting conditions as these are controlled by the display.

Referring to FIG. 35, A more rigorous test for contrast sensitivity also depends on the spatial frequency of the presented stimulus. An example of such a test is the sine-wave grating test, FIG. 35, in which a set of gratings in different spatial frequencies and different contrasts are presented to the subject.

The subject is then requested for example to indicate the orientation of the gratings. Blank images can be incorporated for further indication. The results of the test are then plotted as contrast-frequency graph, FIG. 36.

Color Vision

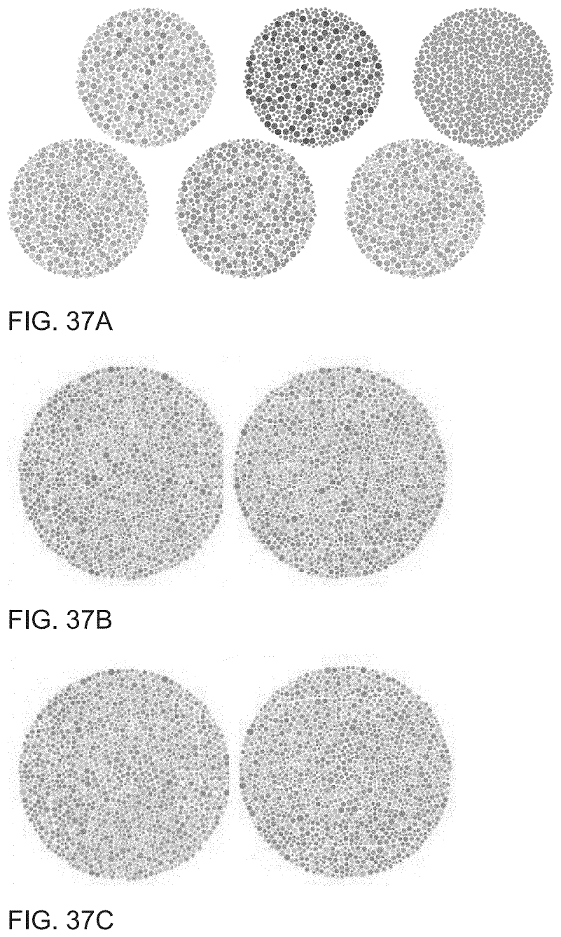

Referring to FIGS. 37A to 37C, color vision tests, as the name implies measure the ability of an individual to see and distinguish color. The most commonly used color vision test is the Ishihara plate test. In this test numerals of different color are composed of circles drawn among other circles of the background color. The circles vary in color to perform various contrasts (mainly red-green, but other combinations are available) while the brightness and contrast of the circles vary between tests for fine tuning the test. FIG. 37A to C present some examples of Ishihara color vision test.

Various types of color blindness could be tested by changing the colors of the letter and the color of the background. A deeper analysis could also include the color saturation and contrast. The implementation in this invention has the advantage of optimal lighting conditions and exact color definition as these are controlled by the display.

FoV

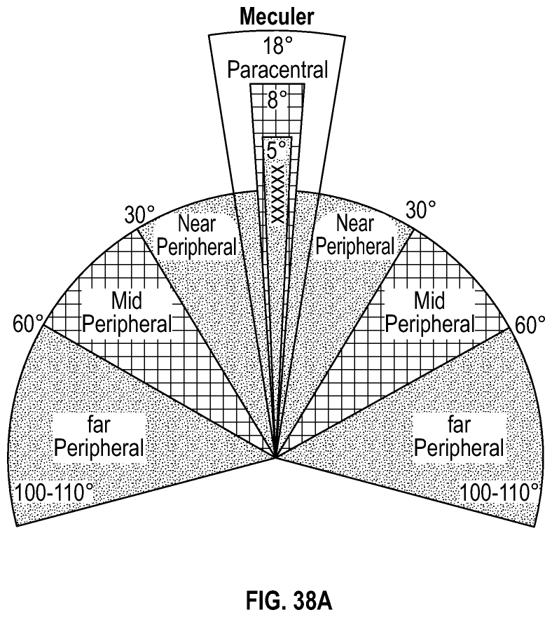

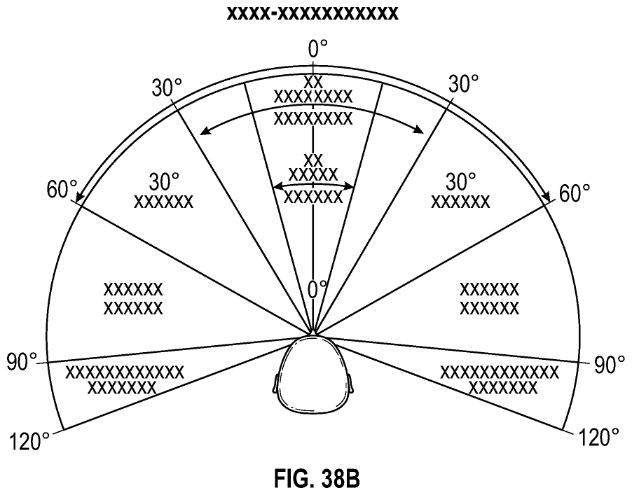

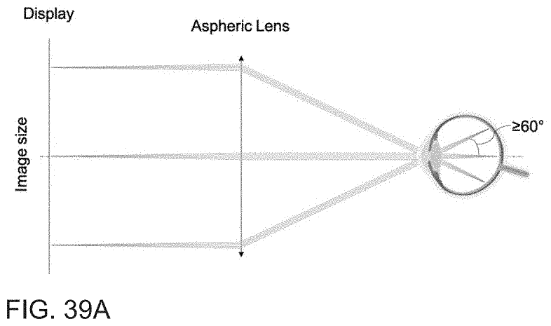

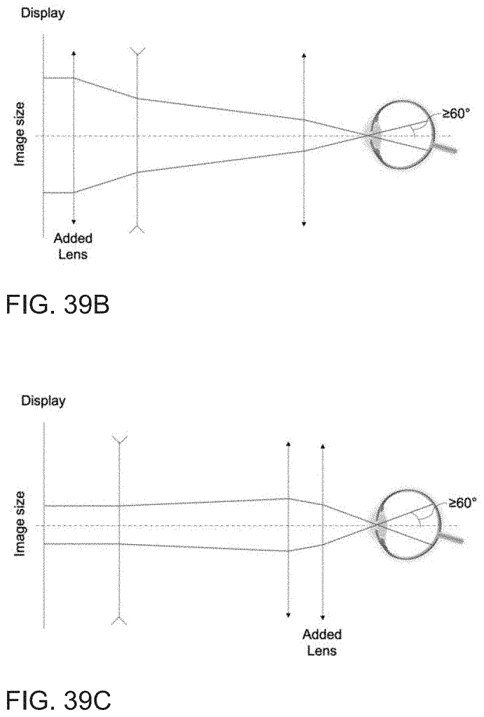

Referring to FIGS. 38A and 39B, an example of the FoV test is based on the automatic perimetry test that requires a very large FoV of the used device (>120 deg and even >180 deg). This field of view is based on the understanding of a user's field of view as described in FIGS. 38A and 38B.

A proposed embodiment of the invention design is presented in FIG. 39A.

Another implementation of the proposed invention is based on the optical train of FIG. 27 where a lens is either added between the screen and the first lens or between the eye and the second lens (FIGS. 39 B and 39 C). The final field of view could be 120 degrees for example.

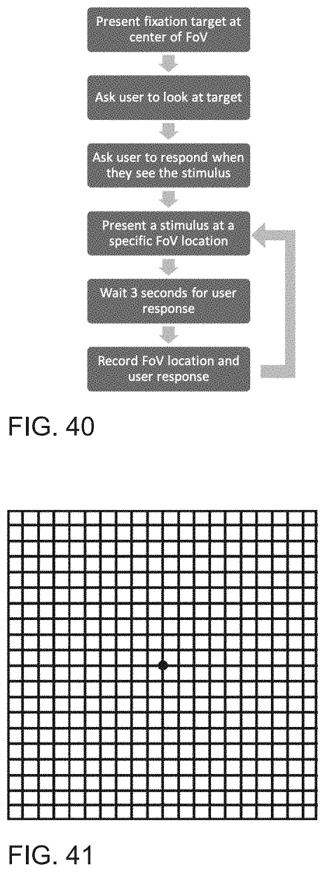

The test itself is quite simple: a stimulus in the form of an illuminated symbol is presented in different locations in the user's FoV and the user is requested to indicate whether they can see it. The symbol could be of different shapes, sizes, colors and brightness. Test characteristics need to be taken into consideration and include beside the symbol itself, the contrast to the background, the stimulus frequency and duration. FIG. 40 presents an implementation of such a test procedure.

Another implementation of the proposed invention includes an optical system of FIG. 27 with a relatively limited field of view. The test is then constructed such that the user's field of view is tested in segments. This is done by performing the procedure of FIG. 40 for a fixation point at different locations of the screen. This will effectively enable tripling the field of view measurement in any direction.

Referring to FIG. 41, Another implementation of a FoV test measures central field of view defects and is called the Amsler Grid (FIG. 40). In this test the user focuses on the point in the middle of the grid for each eye separately and indicates any distorted, faded, or partially missing lines around it.

Depth Perception (Stereopsis)

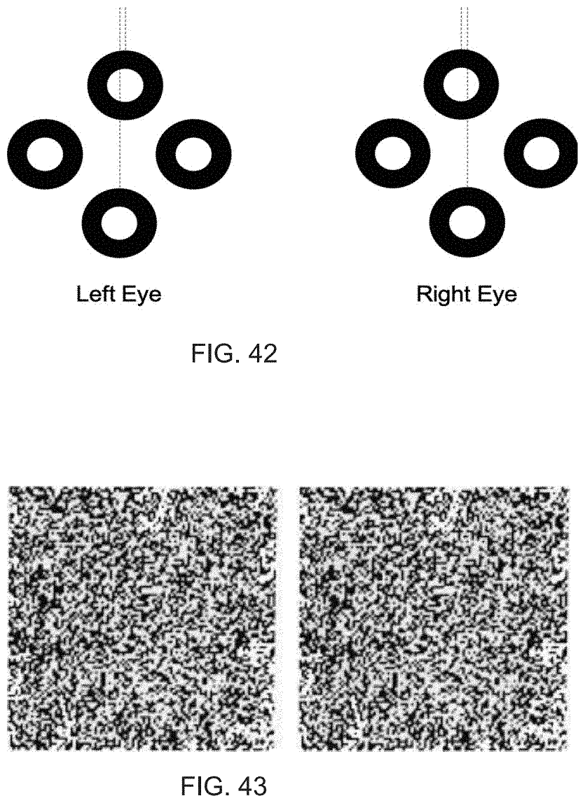

The simplest form of the test would be presentation of four similar symbols (FIG. 42) in a rhombus configuration.

One of the images would be presented at a different depth than the other three (using the methods presented above for 3D vision). The user will then be required to indicate which of the images is the one closer. Multiple sets will be repeated with the distance contrast between the shapes different for each set (different angular disparity for example between 30 arcsec and 1200 arcsec).

Referring to FIG. 43, another form of depth perception test is the random dot test in which an image of random dots with features that could be detected using stereopsis are presented to the user (example of a H shape is presented in FIG. 43). The test could be designed to include Landolt C or tumbling E and the user could then be requested to indicate the direction of the given cue. Other tests could also be implemented including for example the Titmus stereotest.

Frequency Measurement

This test allows for indication of potential nerve damage (including for example early glaucoma) and other visual impairments.

In an embodiment of this test, two bars are presented to the user. These flicker at different frequencies and the user is requested to indicate how many bars they see. At certain frequencies, users with visual and neural problems will not be able to see the lines or will see four lines instead of two.

The above detailed description of embodiments of the invention is not intended to be exhaustive or to limit the invention to the precise form disclosed above. While specific embodiments of, and examples for, the invention are described above for illustrative purposes, various equivalent modifications are possible within the scope of the invention, as those skilled in the relevant art will recognize. For example, while steps are presented in a given order, alternative embodiments may perform routines having steps in a different order. The teachings of the invention provided herein can be applied to other systems, not only the systems described herein. The various embodiments described herein can be combined to provide further embodiments. These and other changes can be made to the invention in light of the detailed description.

All the above references and U.S. patents and applications are incorporated herein by reference. Aspects of the invention can be modified, if necessary, to employ the systems, functions and concepts of the various patents and applications described above to provide yet further embodiments of the invention.

These and other changes can be made to the invention in light of the above detailed description. In general, the terms used in the following claims, should not be construed to limit the invention to the specific embodiments disclosed in the specification, unless the above detailed description explicitly defines such terms. Accordingly, the actual scope of the invention encompasses the disclosed embodiments and all equivalent ways of practicing or implementing the invention under the claims.

While certain aspects of the invention are presented below in certain claim forms, the inventors contemplate the various aspects of the invention in any number of claim forms.

What is claimed is:

1. A system for presenting visual images to an optical system, the system comprising:
a) a housing (200);
b) the housing containing a pair of lens tubes (250)
c) each lens tube in visual communication with a second lens (360);
d) a first lens (320) in visual communication with the second lens, the first lens comprising a front surface and the first lens comprising a back surface
e) a front cover (260) configured to accommodate a screen (405) of an electronic device such that the screen of the electronic device is the optical plane the front surface of the first lens;
f) the lenses of the first lens tube configured to produce a horizontal angular disparity in an image presented to the optical system as compared to the image presented by the lenses of the second lens tube.

2. A system for presenting visual images to an optical system, the system comprising:
a) a housing (200);
b) the housing containing a pair of lens tubes (250);
c) each lens tube in visual communication with a second lens, the second lens comprising a variable lens system to allow for refraction correction; the second lens disposed adjacent to the optical system;
d) a first lens in visual communication with the second lens, the first lens comprising a front surface and the first lens comprising a back surface; and
e) a front cover (260) configured to accommodate a screen (405) of an electronic device such that the screen of the electronic device is in the optical plane of the front surface of the first lens.

3. The system of claim 2 wherein the second lens comprises a liquid lens.

4. The system of claim 2 wherein the second lens is a zoom lens comporting a Stokes equation for controlling cylinder and axis adjustments.

5. The system of claim 2 wherein the second lens is an elastic deformable lens.

6. The system of claim 1 wherein a field of view adjustment lens is disposed between the first lens and the screen of the electronic device.

7. The system of claim 1 wherein a field of view adjustment lens is disposed between the second lens and the optical system.

8. The system of claim 6 used to test the field of view for an optical system.

9. The system of claim 1 wherein the screen comprises a liquid crystal display built in to the front cover.

10. The system of claim 1 having an object plane disposed adjacent to the first lens, the object plane selected from the group comprising a liquid crystal display, organic light emitting diode array and/or light emitting diode array.

11. The system of claim 1 wherein a test figures are presented to the optical system, with the test figures rotated and presented in descending sizes.

12. The system of claim 1 wherein color vision test figures are disposed within the optical plane of the front surface of the first lens.

13. The system of claim 1 wherein contrast sensitivity figures are disposed within the optical plane of the front surface of the first lens.

14. The system of claim 1 wherein a fixation point is disposed upon an Amsler Grid to measure the field of view of an optical system.

15. The system of claim 1 wherein the lens system comports to a field of view test, the test selected from the group comprising: confrontational visual filed testing, static automated perimetry and kinetic perimetry.

16. The system of claim 1 wherein different images are disposed within the optical plane of the first lens tube and second lens tube to test the depth perception of an optical system.

17. The system of claim 1 presenting a plurality of symbols to the optical plane of the first and second lens tubes with each symbol flickering at a different frequency.

18. A method of presenting visual images to an optical system, the method comprising the steps of:
a) using a housing (200); the housing containing a pair of lens tubes (25)
b) disposing each lens tube to be in visual communication with a second lens (360);
c) disposing a first lens (320) to be in visual communication with the second lens, the first lens comprising a front surface and the first lens comprising a back surface;
d) disposing a front cover (260) configured to accommodate a screen (405) of an electronic device such that the screen of the electronic device is the optical plane of the front surface of the first lens;
e) disposing the lenses of the first lens tube to produce a horizontal angular disparity in an image presented to the optical system as compared to the image presented by the lenses of the second lens tube.
